(19) United States
(12) Patent Application Publication
(10) Pub. No.: US 2025/0341523 A1
(43) Pub. Date: Nov. 6, 2025

(54) Reagent Kit, Staining Method, and Application for Dual Immunohistochemistry Combined with Elastin Fiber Staining

(71) Applicants: Feng ZHANG, Zhejiang (CN); Xingchang REN, Zhejiang (CN)

(72) Inventors: Feng ZHANG, Zhejiang (CN); Xingchang REN, Zhejiang (CN)

(21) Appl. No.: 18/945,455

(22) Filed: Nov. 12, 2024

(30) Foreign Application Priority Data
May 6, 2024 (CN) 2024105442651

(51) Int. Cl. G01N 33/58

(52) U.S. Cl. CPC G01N 33/581; G01N 2333/908; G01N 2333/916; G01N 2474/20

(57) ABSTRACT

A reagent kit, staining method, and application for combining dual immunohistochemistry with elastin fiber staining. The reagent kit includes a combination of primary antibodies, consisting of anti-CK7 and anti-CD34 monoclonal antibodies from different species, such as rabbit anti-human CK7 monoclonal antibody and mouse anti-human CD34 monoclonal antibody. By selecting CK7 and CD34 as the primary antibody combination, the growth pattern of tumor epithelium can be accurately indicated, and the proliferation and thickening of alveolar septal fibrous tissue caused by tumors can be clearly revealed, as opposed to structural changes of alveoli induced by other factors. Combined with independent Victoria blue staining, the elastin fibers of the walls of arteries and veins, as well as the pleura of the lungs, can be clearly visualized, aiding in the assessment of the integrity of elastin fibers on the lung surface and in determining the extent of tumor infiltration.

### TECHNICAL FIELD

The present invention relates to the field of clinical pathology testing, specifically a reagent kit, staining method, and application for dual immunohistochemistry combined with elastin fiber staining.

### BACKGROUND OF THE INVENTION

Immunohistochemistry (IHC), also known as immunocytochemistry staining, is one of the most important platform technologies for pathological diagnosis, differential diagnosis, and the identification of tumor-specific molecular targets. This technique is based on the antigen-antibody reaction principle in immunology, where a chromogenic agent labeled on the antibody undergoes a chemical reaction, allowing for the localization, qualitative, and relative quantitative study of antigens within tissue cells. Traditional immunohistochemistry techniques typically target a single antigen for staining. However, considering the “three early” principles of tumor management—early detection, early diagnosis, and early treatment—as well as the precious nature of small tissue lesion samples, it has become increasingly important to develop techniques capable of detecting multiple antigen targets and performing elastin fiber staining on a single tissue section. This has led to the advancement of multicolor immunohistochemistry, which allows for the observation of multiple antigens on the same tissue section, greatly facilitating the understanding of the relationships between different protein expressions. This has proven to be particularly important in pathological diagnosis for diseases of the prostate, breast, digestive tract, and other organs.

Since the 2010 update of the multidisciplinary classification of lung adenocarcinoma, clinical and pathological diagnostic standards have significantly improved, with diagnostic criteria becoming more defined and refined. However, in practice, issues with diagnostic consistency remain prominent, severely affecting patient treatment and prognosis. In traditional immunohistochemistry using cytokeratin 7 (CK7) and elastin fiber double staining, the contrast between the brownish staining of CK7 in the cytoplasm of alveolar epithelial cells and lung adenocarcinoma cells and the deep blue wavy staining of elastin fibers with Victoria blue is not always clear. Additionally, the display of the alveolar wall structure is not sufficiently distinct, making diagnosis challenging.

### BRIEF SUMMARY OF THE INVENTION

### (1) Technical Problem Solved

In response to the shortcomings of existing technology, this invention provides a reagent kit, staining method, and application for dual immunohistochemistry combined with elastin fiber staining, addressing the issues presented in the background technology.

### (2) Technical Solution

To achieve the above objectives, the invention is realized through the following technical solutions:

According to the first aspect of the invention, a reagent kit for dual immunohistochemistry combined with elastin fiber staining is provided. This reagent kit includes a combination of primary antibodies, where the combination consists of monoclonal antibodies against CK7 and CD34 from different species.

Preferably, the combination of primary antibodies includes rabbit anti-human CK7 monoclonal antibody and mouse anti-human CD34 monoclonal antibody.

Preferably, the reagent kit further comprises the following components: hydrogen peroxide blocking agent, secondary antibody reagent, DAB chromogenic solution, Fast Red series chromogenic agent, hematoxylin counterstain solution, bluing reagent, Victoria blue staining solution, 95% ethanol, and absolute ethanol.

Preferably, the secondary antibody reagent includes:
1) A secondary antibody polymer reagent labeled with horseradish peroxidase from one species and a secondary antibody polymer reagent labeled with alkaline phosphatase from another species; or
2) A secondary antibody reagent composed of a mixture of horseradish peroxidase-labeled secondary antibody polymer and alkaline phosphatase-labeled secondary antibody polymer from different species.

More preferably, the Fast Red series chromogenic agent includes a Fast Red precursor, Fast Red modifier, and Fast Red substrate, with a mass ratio of 1:1:1 for the Fast Red precursor, Fast Red modifier, and Fast Red substrate.

The DAB chromogenic solution includes DAB chromogen and DAB buffer, with a mass ratio of 1:1 for the DAB chromogen and DAB buffer.

The DAB buffer contains imidazole hydrochloride buffer and hydrogen peroxide.

According to the second aspect of the invention, a staining method for dual immunohistochemistry combined with elastin fiber staining is provided. This method involves performing routine slide baking, deparaffinization, rehydration, antigen heat retrieval, and blocking of endogenous peroxidase on tissue sections to be tested. The combination of primary antibodies is then used to incubate the tissue sections, followed by incubation with the secondary antibody reagent. The Fast Red series chromogenic agent is used for staining, followed by incubation with DAB chromogenic solution. After hematoxylin counterstaining and bluing, Victoria blue staining is applied directly, followed by routine dehydration, clearing, and coverslipping.

A specific method for dual immunohistochemistry combined with elastin fiber staining includes the following steps:
(1) Perform routine antigen heat retrieval on tissue sections after slide baking, deparaffinization, and rehydration.
(2) Apply hydrogen peroxide blocking agent to the tissue sections to block endogenous peroxidase.
(3) Incubate the tissue sections with the primary antibodies, followed by incubation with the secondary antibody reagent. The primary antibodies include rabbit anti-human CK7 monoclonal antibody and mouse anti-human CD34 monoclonal antibody.
(4) Incubate the tissue sections with the Fast Red series chromogenic agent, followed by incubation with DAB chromogenic solution.
(5) Rinse the tissue sections with pure water, incubate with hematoxylin staining solution, then incubate with bluing reagent, and finally incubate with Victoria blue staining solution.
(6) Sequentially immerse the tissue sections in 95% ethanol and absolute ethanol, then air dry at room temperature.

1. According to the third aspect of the invention, the reagent kit or staining method described above is applied in pathological tissue staining and diagnosis.

### (3) Beneficial Effects

The invention provides a reagent kit, staining method, and application for dual immunohistochemistry combined with elastin fiber staining, offering the following beneficial effects:
1. The reagent kit for dual immunohistochemistry combined with elastin fiber staining provided in this invention utilizes CK7 and CD34 as primary antibody targets, along with standalone Victoria blue staining. CK7 stains red in the cytoplasm of alveolar epithelial cells and tumor glandular epithelial cells in the tissue sections, while CD34 stains brown in the endothelial cells within the alveolar septa. This enables clear visualization of whether the tumor epithelium is growing along the alveolar wall, allowing effective differentiation between alveolar septal fibrosis caused by the tumor, alveolar collapse and structural obscuration due to various factors, and tumor infiltration leading to alveolar destruction and disappearance. Victoria blue staining results in a deep blue-violet wavy coloration of the elastin fibers in the arterial and venous walls, alveolar septa, and pleura, which helps assess the extent of damage to the inner and outer elastin fibers on the lung surface.
2. The reagent kit provided by this invention allows for the simultaneous detection of CK7, CD34, and the expression of Victoria blue stain on a single tissue section. This significantly reduces the amount of sample required and facilitates observation of the relationship and co-localization of target proteins as well as tissue morphology. It effectively enhances the accuracy of pathological results, providing objective, readable data, and improves diagnostic consistency.

The staining method for dual immunohistochemistry combined with elastin fiber staining offered by this invention eliminates the need for sequential incubation of individual primary antibodies, thus saving experimental time. The elastin fiber staining step only uses Victoria blue dye, reducing the complexity of the procedure and further saving experimental time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: The result of CD34/CK7 immunohistochemistry combined with Victoria blue staining on the first tissue section using the reagent kit from Example 1 of this invention.

FIG. 2: The result of CD34/CK7 immunohistochemistry combined with Victoria blue staining on the second tissue section using the reagent kit from Example 1 of this invention.

FIG. 3: The result of CD34/CK7 immunohistochemistry combined with Victoria blue staining on the third tissue section using the reagent kit from Example 1 of this invention.

FIG. 4: The result of CD34/CK7 immunohistochemistry combined with Victoria blue staining on the fourth tissue section using the reagent kit from Example 1 of this invention.

FIG. 5: The result of CD31 immunohistochemistry staining on the tissue section using the reagent kit from Comparative Example 1 of this invention.

FIG. 6: The result of TTF-1 immunohistochemistry staining on the tissue section using the reagent kit from Comparative Example 2 of this invention.

FIG. 7: The result of NapsinA immunohistochemistry staining on the tissue section using the reagent kit from Comparative Example 3 of this invention.

FIG. 8: The result of P63/CD34/CK7 immunohistochemistry staining on the tissue section using the reagent kit from Comparative Example 4 of this invention.

FIG. 9: The result of P40/CD34/CK7 immunohistochemistry staining on the tissue section using the reagent kit from Comparative Example 5 of this invention.

FIG. 10: The result of CD34/CK7 immunohistochemistry staining on the tissue section using the reagent kit from Comparative Example 6 of this invention.

FIG. 11: The result of CD34/CK7 immunohistochemistry combined with special elastin fiber staining on the tissue section using the reagent kit from Comparative Example 7 of this invention.

### DETAILED DESCRIPTION OF THE INVENTION

To better explain the content of the invention, the following detailed examples are provided.

The invention offers a reagent kit for dual immunohistochemistry combined with elastin fiber staining, which includes the following components: hydrogen peroxide blocking agent, combination of primary antibodies, secondary antibody reagent, DAB chromogenic solution, Fast Red series chromogenic agent, hematoxylin counterstain solution, bluing reagent, Victoria blue staining solution, 95% ethanol, and absolute ethanol. The combination of primary antibodies includes rabbit anti-human CK7 monoclonal antibody and mouse anti-human CD34 monoclonal antibody, while the secondary antibody reagent includes horseradish peroxidase-labeled anti-mouse secondary antibody polymer and/or alkaline phosphatase-labeled anti-rabbit secondary antibody polymer.

In one embodiment, the Fast Red series chromogenic agent includes Fast Red precursor, Fast Red modifier, and Fast Red substrate, with a mass ratio of 1:1:1 for the Fast Red precursor, Fast Red modifier, and Fast Red substrate.

In one embodiment, the DAB chromogenic solution includes DAB chromogen and DAB buffer, with a mass ratio of 1:1 for the DAB chromogen and DAB buffer.

In one embodiment, the DAB buffer includes imidazole hydrochloride buffer and hydrogen peroxide.

As a specific staining method for dual immunohistochemistry combined with elastin fiber staining, the reagent kit described above is used, and can be applied manually, via a fully automated immunohistochemistry stainer, or with a fully automated special stainer, following these steps:

Step-by-Step Staining Procedure:
1. Place the tissue sections to be tested in an oven at 68° C. for 60 minutes for baking, then transfer the sections into a water-based deparaffinization solution and heat in a water bath at 75° C. for 15 minutes for deparaffinization and rehydration.
2. Place the tissue sections in an antigen retrieval solution, heat at 95-100° C. for 20-30 minutes for antigen retrieval, then cool to room temperature. Take out the tissue sections and gently rinse twice with distilled water, with each rinse lasting 3 minutes.
3. Add 100 μL of hydrogen peroxide blocking agent to the tissue sections and incubate for 10 minutes to block endogenous peroxidase. Gently rinse three times with buffer, with each rinse lasting 3 minutes.
4. Add 100 μL of the primary antibody combination to the tissue sections, incubate at room temperature for 60 minutes, and then gently rinse three times with buffer, with each rinse lasting 3 minutes.
5. Add 100 μL of the secondary antibody reagent to the tissue sections, incubate at room temperature for 30 minutes, and then gently rinse three times with buffer, with each rinse lasting 3 minutes.
6. Add 100-200 μL of the Fast Red series chromogenic agent to the tissue sections, incubate at room temperature for 10 minutes, and then gently rinse three times with buffer, with each rinse lasting 3 minutes.
7. Add 100-200 μL of DAB chromogenic solution to the tissue sections, incubate at room temperature for 5 minutes, and then gently rinse off the DAB solution with distilled water to stop the chromogenic reaction.
8. Add 100-200 μL of hematoxylin staining solution to the tissue sections and incubate for 10-30 seconds, followed by a rinse with distilled water.
9. Add 100-200 μL of bluing reagent to the tissue sections and incubate for 2-3 minutes, followed by a rinse with distilled water, and air dry at room temperature.
10. Add 3 mL of Victoria blue staining solution to the tissue sections for counterstaining, and incubate for 2 hours.
11. Immerse the tissue sections in 95% ethanol three times, with each immersion lasting 20 seconds, followed by a 3-second immersion in absolute ethanol. Air dry at room temperature, then coverslip using neutral mounting medium and a coverslip.

The following description explains the reagent kit, staining method, and application of dual immunohistochemistry combined with elastin fiber staining according to specific embodiments of the present invention.

### EXAMPLE 1

This example provides a reagent kit for dual immunohistochemistry combined with elastin fiber staining, which includes the following components:
1. Hydrogen peroxide blocking agent: 3% hydrogen peroxide, sodium azide, and inorganic salts;
2. Primary antibody combination: rabbit anti-human CK7 monoclonal antibody and mouse anti-human CD34 monoclonal antibody;
3. Secondary antibody reagent: horseradish peroxidase-labeled anti-mouse secondary antibody polymer and alkaline phosphatase-labeled anti-rabbit secondary antibody polymer;
4. DAB chromogenic solution: DAB chromogen and DAB buffer in a 1:1 mass ratio. The DAB buffer contains imidazole hydrochloride buffer and hydrogen peroxide;
5. Fast Red series chromogenic agent: Fast Red precursor, Fast Red modifier, and Fast Red substrate in a 1:1:1 mass ratio. The Fast Red precursor contains 2% hydrochloric acid and sodium nitrite. The Fast Red substrate contains naphthol AS-BI phosphate, Tris, and dimethylformamide;
6. Hematoxylin counterstain solution: hematoxylin, absolute ethanol, aluminum potassium sulfate, mercuric oxide, and glacial acetic acid;
7. Bluing reagent: 0.5% ammonia water;
8. Victoria blue staining solution: Victoria blue, dextrin, resorcinol, and ferric chloride;
9. 95% ethanol;
10. Absolute ethanol.

The staining method using this reagent kit includes the following steps:
1. Place the tissue sections in an oven at 68° C. for 60 minutes for baking, then transfer the sections into a water-based deparaffinization solution and heat in a water bath at 75° C. for 15 minutes for deparaffinization and rehydration.
2. Place the tissue sections in antigen retrieval solution (EDTA, Tris, Triton X-100, Tween 20) and heat at 95-100° C. for 20-30 minutes. Cool to room temperature, take out the tissue sections, and gently rinse twice with distilled water, with each rinse lasting 3 minutes.
3. Add 100 μL of hydrogen peroxide blocking agent (3% hydrogen peroxide, sodium azide, inorganic salts) to the tissue sections, incubate for 10 minutes to block endogenous peroxidase, and gently rinse three times with buffer (Tris-HCl, Tris, NaCl, Triton X-100), with each rinse lasting 3 minutes.
4. Add 100 μL of the primary antibody combination (rabbit anti-human CK7 and mouse anti-human CD34 monoclonal antibodies) to the tissue sections, incubate at room temperature for 60 minutes, and then gently rinse three times with buffer (Tris-HCl, Tris, NaCl, Triton X-100), with each rinse lasting 3 minutes.
5. Add 100 μL of the secondary antibody reagent (horseradish peroxidase-labeled anti-mouse secondary antibody polymer and alkaline phosphatase-labeled anti-rabbit secondary antibody polymer) to the tissue sections, incubate at room temperature for 30 minutes, and then gently rinse three times with buffer (Tris-HCl, Tris, NaCI, Triton X-100), with each rinse lasting 3 minutes.
6. Add 100-200 μL of the Fast Red series chromogenic agent to the tissue sections, incubate at room temperature for 10 minutes, and gently rinse three times with buffer (Tris-HCl, Tris, NaCl, Triton X-100), with each rinse lasting 3 minutes.
7. Add 100-200 μL of DAB chromogenic solution to the tissue sections, incubate at room temperature for 5 minutes, and gently rinse with distilled water to stop the chromogenic reaction.
8. Add 100 μL of hematoxylin staining solution (hematoxylin, absolute ethanol, aluminum potassium sulfate, mercuric oxide, glacial acetic acid) to the tissue sections, incubate for 10-30 seconds, and rinse with distilled water.
9. Add 100 μL of bluing reagent (0.5% ammonia water) to the tissue sections, incubate for 2-3 minutes, rinse with distilled water, and air dry at room temperature.
10. Add 3 mL of Victoria blue staining solution (Victoria blue, dextrin, resorcinol, ferric chloride) to the tissue sections for counterstaining, and incubate for 2 hours.
11. Immerse the tissue sections in 95% ethanol three times, with each immersion lasting 20 seconds, then immerse them in absolute ethanol for 3 seconds. Air dry at room temperature, and coverslip using neutral mounting medium and a coverslip.

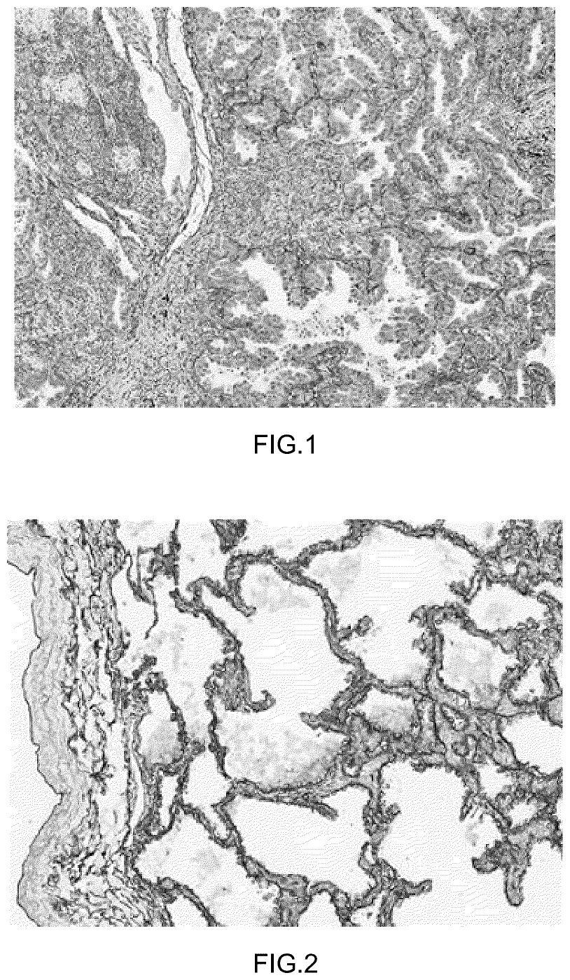

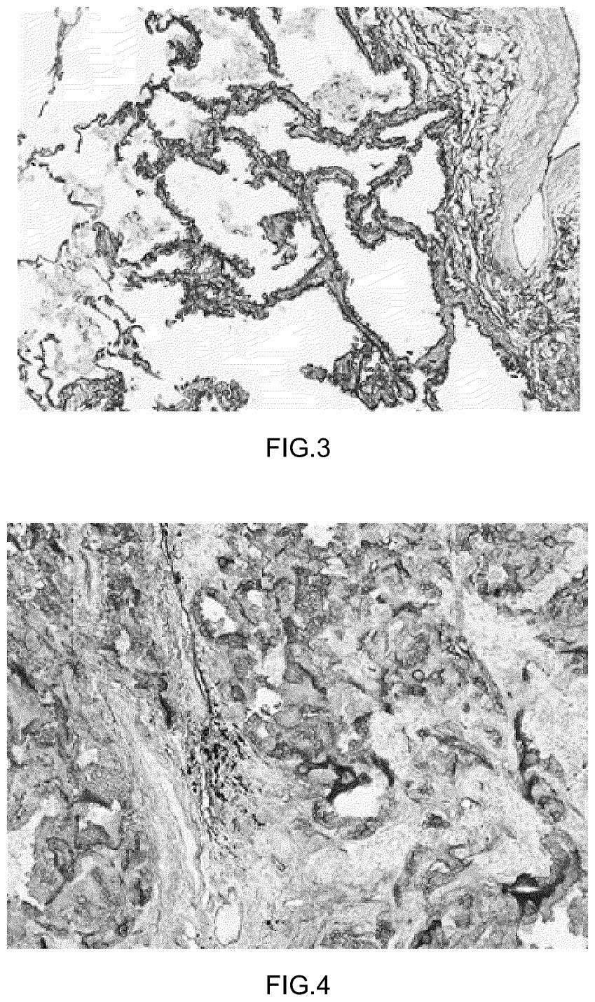

In this example, the above reagent kit was used for staining tests on four different tissue sections. The test results are shown in FIGS. 1 to 4, where the alveolar wall presents a brownish-yellow dense and complete network structure, indicating positive expression of the CD34 antibody in the vascular endothelium, producing a brownish-yellow signal. The tumor alveolar epithelial cells exhibit an enlarged cytoplasm with a red alignment, allowing for the assessment of whether the alveolar wall is present and the relationship between tumor cells and the alveolar wall. This helps objectively determine whether there is attached growth (i.e., carcinoma in situ) or if the alveolar wall structure has disappeared (i.e., invasive changes). At the same time, the blue elastin fibers assist in recognition, eliminating the illusion of invasive changes caused by various factors such as alveolar collapse or thickened alveolar septa. This allows for the objective assessment of conditions such as adenocarcinoma in situ, minimally invasive adenocarcinoma, invasive adenocarcinoma, and aerogenous spread. Additionally, the test enables observation of the infiltration and destruction of visceral pleural elastin fibers, further improving the objective accuracy of the assessment.

### COMPARATIVE EXAMPLE 1

This comparative example provides an immunohistochemistry reagent kit, with the following differences compared to the reagent kit in Example 1: the primary antibody in this comparative example is selected as CD31 antibody, and the secondary antibody is selected as HRP-labeled secondary antibody polymer. It does not contain the Fast Red series chromogenic agent or Victoria blue staining solution.

The staining method using the immunohistochemistry reagent kit of this comparative example includes the following steps:
1. Place the tissue sections to be tested in an oven at 68° C. for 60 minutes for baking, then transfer the sections into a water-based deparaffinization solution and heat in a water bath at 75° C. for 15 minutes for deparaffinization and rehydration.
2. Place the tissue sections in antigen retrieval solution (EDTA, Tris, Triton X-100, Tween 20) and heat at 95-100° C. for 20-30 minutes. Cool to room temperature, take out the tissue sections, and gently rinse twice with distilled water, with each rinse lasting 3 minutes.
3. Add 100 μL of hydrogen peroxide blocking agent (3% hydrogen peroxide, sodium azide, inorganic salts) to the tissue sections, incubate for 10 minutes to block endogenous peroxidase, and gently rinse three times with buffer (Tris-HCl, Tris, NaCl, Triton X-100), with each rinse lasting 3 minutes.
4. Add 100 μL of CD31 antibody to the tissue sections, incubate at room temperature for 60 minutes, and then gently rinse three times with buffer (Tris-HCl, Tris, NaCl, Triton X-100), with each rinse lasting 3 minutes.
5. Add 100 μL of reaction enhancer to the tissue sections, incubate at room temperature for 10 minutes, and gently rinse three times with buffer (Tris-HCl, Tris, NaCl, Triton X-100), with each rinse lasting 3 minutes. Then, add 100 μL of HRP-labeled secondary antibody polymer, incubate at room temperature for 10 minutes, and gently rinse three times with buffer (Tris-HCl, Tris, NaCl, Triton X-100), with each rinse lasting 3 minutes.
6. Add 100 μL of DAB chromogenic solution to the tissue sections, incubate at room temperature for 5 minutes, and gently rinse with distilled water to stop the chromogenic reaction.
7. Add 100 μL of hematoxylin staining solution (hematoxylin, absolute ethanol, aluminum potassium sulfate, mercuric oxide, glacial acetic acid) to the tissue sections, incubate for 10-30 seconds, and rinse with distilled water.
8. Add 100 μL of bluing reagent (0.5% ammonia water) to the tissue sections, incubate for 2-3 minutes, rinse with distilled water, and air dry at room temperature.
9. Immerse the tissue sections in 95% ethanol three times, with each immersion lasting 20 seconds, then immerse them in absolute ethanol for 3 seconds. Air dry at room temperature, and coverslip using neutral mounting medium and a coverslip.

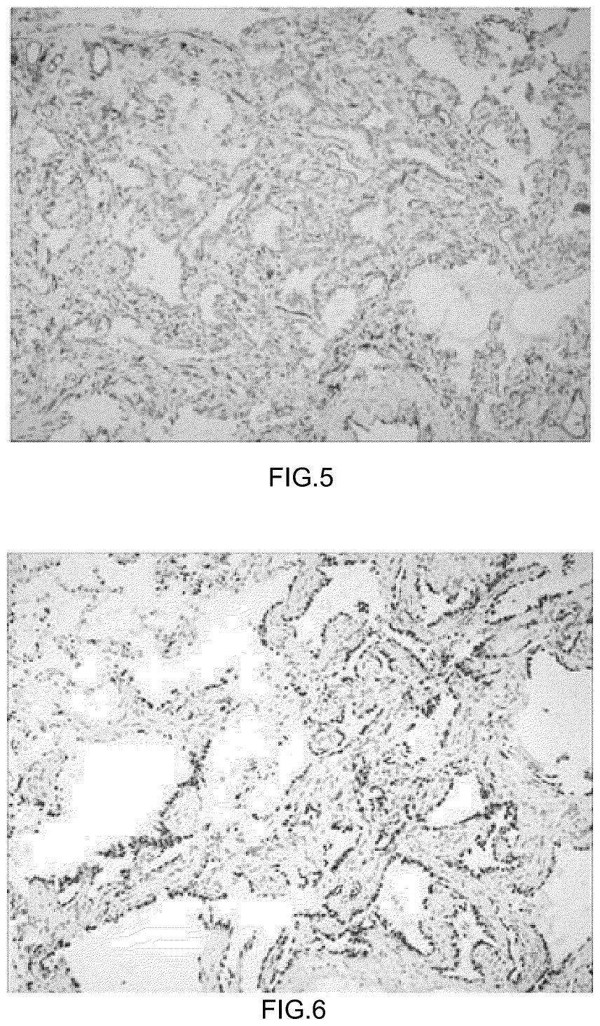

In the staining test using the reagent kit described above, the test results are shown in FIG. 5. Relatively larger alveolar wall capillaries are stained by CD31, with most positive blood vessels being recognizable under a conventional microscope. However, the capillary network of the alveolar walls is not sufficiently displayed, and the alveolar septa are not clearly visible.

### COMPARATIVE EXAMPLE 2

This comparative example provides an immunohistochemistry reagent kit, with the following differences compared to the reagent kit in Example 1: the primary antibody in this comparative example is selected as TTF-1 antibody, and the secondary antibody is selected as HRP-labeled secondary antibody polymer. It does not contain the Fast Red series chromogenic agent or Victoria blue staining solution.

The staining test method for this comparative example is the same as that in Comparative Example 1, with the difference being that in Step 4, TTF-1 antibody is used as the primary antibody.

The staining results of this comparative example are shown in FIG. 6. TTF-1 does not display continuous alveolar epithelium, making it impossible to determine the presence or absence of attached growth.

### COMPARATIVE EXAMPLE 3

This comparative example provides an immunohistochemistry reagent kit, with the following differences compared to the reagent kit in Example 1: the primary antibody in this comparative example is selected as NapsinA antibody, and the secondary antibody is selected as HRP-labeled secondary antibody polymer. It does not contain the Fast Red series chromogenic agent or Victoria blue staining solution.

The staining test method for this comparative example is the same as that in Comparative Example 1, with the difference being that in Step 4, NapsinA antibody is used as the primary antibody.

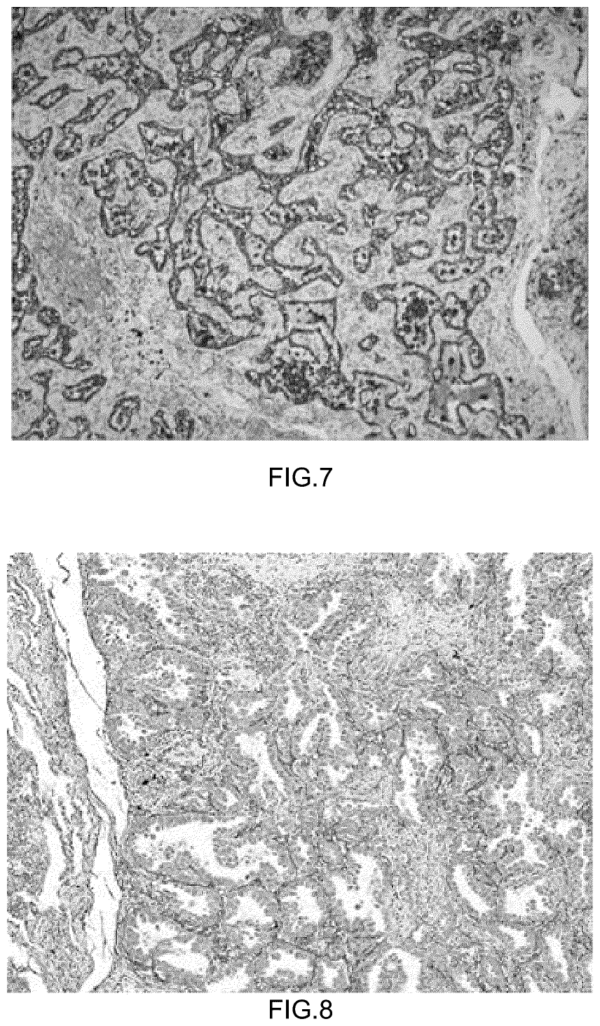

The staining results of this comparative example are shown in FIG. 7. NapsinA displays a complex structure, and the result suggests that adenocarcinoma in situ may appear as invasive changes, making it difficult for the observer to accurately assess the actual situation.

### COMPARATIVE EXAMPLE 4

This comparative example provides an immunohistochemistry reagent kit, with the following differences compared to the reagent kit in Example 1: the primary antibody combination in this comparative example includes P63, CD34, and CK7, and does not contain Victoria blue staining solution.

The staining method for this comparative example is the same as that in Example 1, with the difference being that the primary antibody combination in Step 4 is replaced by P63, CD34, and CK7, and Step 10 is not performed.

The staining test results are shown in FIG. 8. The P63 antibody shows positive expression in the cell nucleus, presenting a brownish-yellow color, which is observed in residual bronchiolar walls or bronchial adenomas, but not in lung adenocarcinoma-related lesions. The CD34 antibody shows positive expression in the vascular endothelium, presenting a brownish-yellow signal, and can display the capillary network of the alveolar walls, highlighting the presence or absence of the alveolar wall. The CK7 antibody shows a red signal in the cytoplasm of the enlarged tumor alveolar epithelial cells, but in some areas, the structure is complex, making it difficult to determine whether there is attached growth. The inclusion of P63 in this combination is valuable for identifying benign bronchial adenoma lesions, but it may have a negative impact on the stratified diagnosis of lung adenocarcinoma to some extent.

### COMPARATIVE EXAMPLE 5

This comparative example provides an immunohistochemistry reagent kit, with the following differences compared to the reagent kit in Example 1: the primary antibody combination in this comparative example includes P40, CD34, and CK7, and does not contain Victoria blue staining solution.

The staining method for this comparative example is the same as that in Example 1, with the difference being that the primary antibody combination in Step 4 is replaced by P40, CD34, and CK7, and Step 10 is not performed.

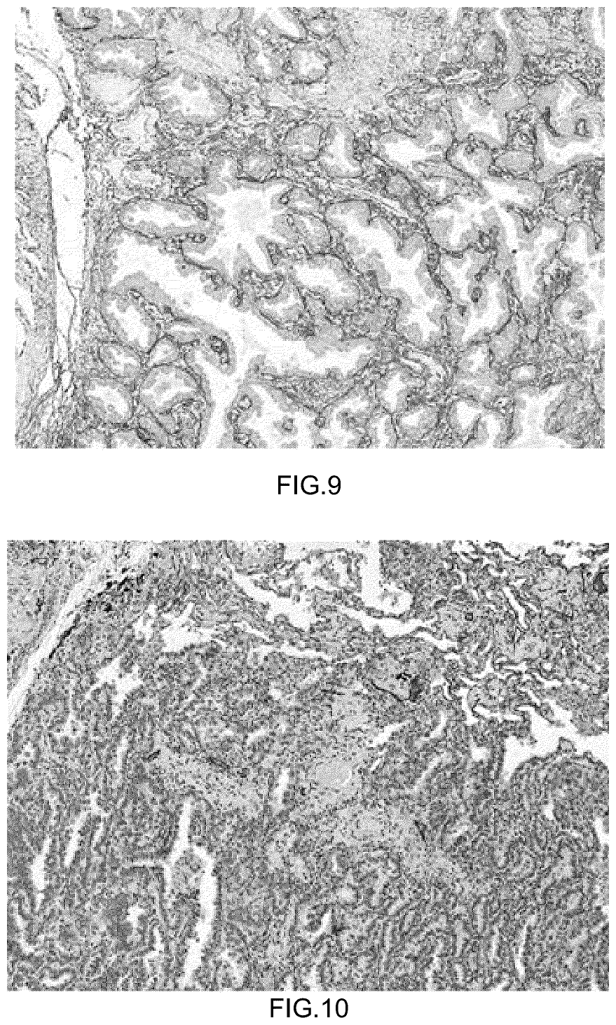

The staining test results are shown in FIG. 9. The P40 antibody shows positive expression in the cell nucleus, presenting a brownish-yellow color, which is observed in residual bronchiolar walls or bronchial adenomas, but not in lung adenocarcinoma-related lesions. The CD34 antibody shows positive expression in the vascular endothelium, presenting a brownish-yellow signal, and can display the capillary network of the alveolar walls, highlighting the presence or absence of the alveolar wall. The CK7 antibody shows a red signal in the cytoplasm of the enlarged tumor alveolar epithelial cells, but in some areas, the structure is complex, making it difficult to determine whether there is attached growth. The inclusion of P40 in this combination is valuable for identifying benign bronchial adenoma lesions, but it may have a negative impact on the stratified diagnosis of lung adenocarcinoma to some extent.

### COMPARATIVE EXAMPLE 6

This comparative example provides an immunohistochemistry reagent kit, with the following difference compared to the reagent kit in Example 1: it does not contain Victoria blue staining solution.

The staining method for this comparative example is the same as that in Example 1, with the difference being that Step 10 is not performed.

The staining test results are shown in FIG. 10. The CD34 antibody shows positive expression in the vascular endothelium, presenting a brownish-yellow signal, which displays the capillary network of the alveolar walls and highlights the presence or absence of the alveolar wall. The CK7 antibody shows a red signal in the cytoplasm of the enlarged tumor alveolar epithelial cells, but in some areas, the structure is complex, making it difficult to determine whether there is attached growth. This combination can display the relationship between the alveolar wall and abnormally proliferating alveolar epithelium, which helps in determining whether the tumor cells exhibit attached growth. It provides a morphological basis for observing and diagnosing the stratification of lung adenocarcinoma. However, the increased focal stromal components still make it somewhat challenging to assess the presence of the alveolar wall.

### COMPARATIVE EXAMPLE 7

This comparative example provides an immunohistochemistry reagent kit, with the following difference compared to the reagent kit in Example 1: it additionally includes potassium permanganate solution and oxalic acid solution.

The staining method using the reagent kit for this comparative example includes the following steps:
1. Place the tissue sections in an oven at 68° C. for 60 minutes for baking, then transfer the sections into a water-based deparaffinization solution and heat in a water bath at 75° C. for 15 minutes for deparaffinization and rehydration.
2. Place the tissue sections in antigen retrieval solution (EDTA, Tris, Triton X-100, Tween 20) and heat at 95-100° C. for 20-30 minutes. Cool to room temperature, take out the tissue sections, and gently rinse twice with distilled water, with each rinse lasting 3 minutes.
3. Add 100 μL of hydrogen peroxide blocking agent (3% hydrogen peroxide, sodium azide, inorganic salts) to the tissue sections, incubate for 10 minutes to block endogenous peroxidase, and gently rinse three times with buffer (Tris-HCl, Tris, NaCl, Triton X-100), with each rinse lasting 3 minutes.
4. Add 100 μL of rabbit anti-human CK7 monoclonal antibody and mouse anti-human CD34 monoclonal antibody to the tissue sections, incubate at room temperature for 60 minutes, and then gently rinse three times with buffer (Tris-HCl, Tris, NaCl, Triton X-100), with each rinse lasting 3 minutes.
5. Add 100 μL of horseradish peroxidase-labeled anti-mouse secondary antibody polymer and alkaline phosphatase-labeled anti-rabbit secondary antibody polymer to the tissue sections, incubate at room temperature for 30 minutes, and gently rinse three times with buffer (Tris-HCl, Tris, NaCl, Triton X-100), with each rinse lasting 3 minutes.
6. Add 100-200 μL of Fast Red series chromogenic agent to the tissue sections, incubate at room temperature for 10 minutes, and gently rinse three times with buffer (Tris-HCl, Tris, NaCl, Triton X-100), with each rinse lasting 3 minutes.
7. Add 100-200 μL of DAB chromogenic solution to the tissue sections, incubate at room temperature for 5 minutes, and gently rinse with distilled water to stop the chromogenic reaction.
8. Add 100 μL of hematoxylin staining solution (hematoxylin, absolute ethanol, aluminum potassium sulfate, mercuric oxide, glacial acetic acid) to the tissue sections, incubate for 10-30 seconds, and rinse with distilled water.
9. Add 100 μL of bluing reagent (0.5% ammonia water) to the tissue sections, incubate for 2-3 minutes, rinse with distilled water, and air dry at room temperature.
10. Add 100 μL of potassium permanganate solution to the tissue sections for oxidation for 15 minutes, rinse with pure water, then add 100 μL of oxalic acid solution for bleaching for 90 seconds, and rinse with pure water.
11. Add 3 mL of Victoria blue staining solution (Victoria blue, dextrin, resorcinol, ferric chloride) to the tissue sections for counterstaining and incubate for 2 hours.
12. Add 95% ethanol to the tissue sections for differentiation for 20 seconds, and rinse with pure water.
13. Add 100 μL of red counterstaining solution to the tissue sections, incubate for 7 minutes, and rinse thoroughly with pure water.
14. Immerse the tissue sections in 95% ethanol three times, with each immersion lasting 20 seconds, then immerse them in absolute ethanol for 3 seconds. Air dry at room temperature, and coverslip using neutral mounting medium and a coverslip.

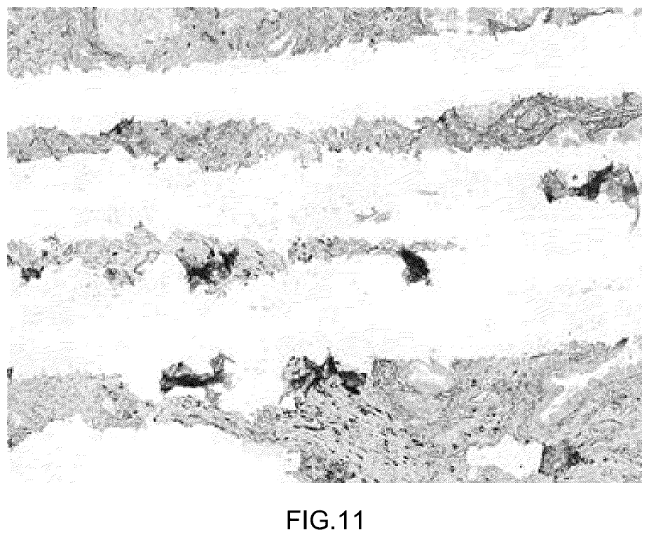

The staining test results are shown in FIG. 11. The CD34 antibody shows positive expression in the vascular endothelium, presenting a brownish-yellow signal, which displays the capillary network of the alveolar walls and highlights the presence or absence of the alveolar wall. The CK7 antibody shows a red signal in the cytoplasm of the enlarged tumor alveolar epithelial cells, but in some areas, the structure is complex, making it difficult to determine whether there is attached growth. This combination can display the relationship between the alveolar wall and the abnormally proliferating alveolar epithelium, which helps in determining whether the tumor cells exhibit attached growth, providing a morphological basis for observing and diagnosing the stratification of lung adenocarcinoma. The display of elastin fibers in areas with increased focal stromal components helps in assessing the presence of the alveolar wall. However, there is significant tissue detachment and pigment deposition, which affects the observer's ability to judge the actual situation. Additionally, the display of elastin fibers is not very clear, and the experimental procedure is rather complex.

The staining test results from Example 1 and Comparative Examples 1 through 7 indicate that the CK7 antigen in the cytoplasm of alveolar and tumor glandular epithelial cells, as well as the CD34 antigen in the endothelial cells within the alveolar septa, can be visualized. CK7 expression typically appears as red staining, while CD34 expression is displayed as brownish staining. This dual-color labeling not only allows for accurate visualization of the tumor epithelium's growth pattern, such as whether there is attached growth, but also helps differentiate between alveolar septal fibrosis caused by tumors and alveolar structural changes induced by other factors. Additionally, Victoria blue staining is used to highlight elastin fibers, making the elastin fibers of the arterial and venous walls, alveolar septa, and visceral pleura of lung tissue clearly visible in a deep blue-violet wavy pattern, particularly the internal and external elastin fibers of the arterial walls and visceral pleura. This staining is critical for assessing the integrity of elastin fibers on the lung surface. By using only Victoria blue for elastin fiber staining, the experimental process is simplified, significantly saving both time and resources, and improving the efficiency and accuracy of the experiment.

Although embodiments of the present invention have been shown and described, it will be understood by those skilled in the art that various changes, modifications, substitutions, and variations can be made to these embodiments without departing from the principles and spirit of the invention. The scope of the present invention is defined by the appended claims and their equivalents.

What is claimed is:

1. A reagent kit for dual immunohistochemistry combined with elastin fiber staining, characterized by: including a combination of primary antibodies, which consists of monoclonal antibodies against CK7 and CD34 from different species.

2. The reagent kit for dual immunohistochemistry combined with elastin fiber staining according to claim 1, characterized by: the combination of primary antibodies includes rabbit anti-human CK7 monoclonal antibody and mouse anti-human CD34 monoclonal antibody.

3. The reagent kit for dual immunohistochemistry combined with elastin fiber staining according to claim 1, characterized by: further comprising the following components: hydrogen peroxide blocking agent, secondary antibody reagent, DAB chromogenic solution, Fast Red series chromogenic agent, hematoxylin counterstain solution, bluing reagent, Victoria blue staining solution, 95% ethanol, and absolute ethanol.

4. The reagent kit for dual immunohistochemistry combined with elastin fiber staining according to claim 3, characterized by: the secondary antibody reagent includes:
1) a secondary antibody polymer reagent labeled with horseradish peroxidase from one species and a secondary antibody polymer reagent labeled with alkaline phosphatase from another species; or
2) a secondary antibody reagent composed of a mixture of horseradish peroxidase-labeled secondary antibody polymer and alkaline phosphatase-labeled secondary antibody polymer from different species.

5. A staining method for dual immunohistochemistry combined with elastin fiber staining, characterized by: performing conventional slide baking, deparaffinization, rehydration, antigen heat retrieval, and blocking of endogenous peroxidase on tissue sections to be tested; incubating the sections with the combination of primary antibodies, followed by incubation with the secondary antibody reagent; performing chromogenic reaction using relevant enzyme substrates, followed by incubation with DAB chromogenic solution; after hematoxylin counterstaining and bluing, directly staining with Victoria blue; and finally performing conventional dehydration, clearing, and coverslipping.

6. Use of the reagent kit according to claim 1 in pathological tissue staining and diagnosis.

7. Use of the reagent kit according to claim 2 in pathological tissue staining and diagnosis.

8. Use of the reagent kit according to claim 3 in pathological tissue staining and diagnosis.

9. Use of the reagent kit according to claim 4 in pathological tissue staining and diagnosis.

10. Use of the staining method according to claim 5 in pathological tissue staining and diagnosis.
